(19) United States
(12) Patent Application Publication
(10) Pub. No.: US 2022/0189756 A1
(43) Pub. Date: Jun. 16, 2022

(54) GLYCAN MASS SPECTROMETRY DATA ANALYZER AND PROGRAM FOR ANALYZING GLYCAN MASS SPECTROMETRY DATA

(71) Applicant: SHIMADZU CORPORATION, Kyoto-shi (JP)

(72) Inventors: Masaki MURASE, Kyoto-shi (JP); Takashi Nishikaze, Kyoto-shi (JP)

(73) Assignee: SHIMADZU CORPORATION, Kyoto-shi (JP)

(21) Appl. No.: 17/644,364

(22) Filed: Dec. 15, 2021

(30) Foreign Application Priority Data
Dec. 16, 2020 (JP) 2020-208470

(51) Int. Cl. H01J 49/00; H01J 49/40; H01J 49/16

(52) U.S. Cl. CPC H01J 49/0036; H01J 49/164; H01J 49/40

(57) ABSTRACT

In a device for analyzing mass spectrum data of a sample containing a sialic-acid-linked glycan subjected to a sialic-acid-linkage-specific derivatization or a molecule modified with the glycan, a glycan information acquisition section acquires a plurality of kinds of glycans having a core structure formed by a plurality of monosaccharide residues of a plurality of kinds, and mass information corresponding to each of those glycans, based on a glycan composition set through a glycan composition setting section. A mass-changing factor setting section allows a setting of mass-changing factors causing a mass change of a glycan. A theoretical glycan mass calculation section determines a theoretical glycan mass-to-charge ratio after the mass change of the glycan occurs, of each glycan based on the mass-changing factors, which include an ion species or salt that is possibly formed on a carboxyl group of a sialic acid in a sialic-acid-linked glycan included in the analysis target and a sialic-acid linkage type in which a mass change corresponding to the derivatization possibly occurs. A mass change calculation section estimates the mass change from the combination of the ion species or the salt and the sialic-acid linkage type.

### TECHNICAL FIELD

The present invention relates to a glycan mass spectrometry data analyzer and a program for analyzing glycan mass spectrometry data.

### BACKGROUND ART

The process of the biosynthesis of proteins, peptides or other biomolecules in living organisms is finely controlled. Therefore, it has been considered that glycosylated biomolecules produced in the biosynthetic process should play it punt roles in vital activities. In recent years, a considerable number of concrete reports at the level of glycan molecules have been made, particularly on the relationship of those molecules with physiology or diseases. With such a background, it has been expected that revealing the structures of glycans which modify biomolecules involved in various processes in vital phenomena will be useful for the elucidation of vital phenomena as well as the drug discovery and diagnosis.

Sialic acids, which are a kind of sugar, have been considered to be an important type of substance for the quality control of proteins, signal transduction in the nervous system, mutual recognition of cells or other aspects of vital actions. In recent years, it has been gradually revealed that the form in which a sialic acid residue is linked to the terminal of a glycan, i.e., the difference in the linkage type of the sialic acid residue, is important for those organic activities. Accordingly, recognizing the linkage type of a sialic acid included in a glycan that modifies biomolecules is important for understanding the function of the glycan in vital activities.

For example, in the case of human beings, α2,3 and α2,6 linkages have been known as the main linkage types of sialic acids. It has been known the linkage type changes with the cancerization of a cell. Accordingly, the idea of using the linkage type as a biomarker or for the quality control of biopharmaceuticals or other purposes has been proposed. However, glycan isomers which only differ from each other in the linkage types of the included sialic acids have no difference in mass. Therefore, it is difficult to determine their linkage types by mass spectrometry, which is a widely used technique for glycan analyses. Another problem is the low level of detection sensitivity and quantitative accuracy, which is due to the fact that sialic acids are unstable since they are easily dissociated from glycans in a mass spectrometric analysis or a pretreatment which precedes the analysis.

To address these problems, techniques for the derivatization of glycans specific to the linkage type of sialic acids have been developed in order to stabilize the structure of the sialic acids and allow different linkage types to be distinguished based on the result of a mass spectrometric analysis. For example, Patent Literature 1 and Non Patent Literature 1 disclose a technique in which the target sample including a glycan is made to react with a dehydration-condensation agent containing an amine (e.g., isopropyl amine) and a carbodiimide to produce a lactone as a derivative of a glycan with α2,3-linked sialic acids as well as an amide as a derivative of a glycan with α2,6-linked sialic acids. Those documents also disclose other derivatization techniques.

The composition and structure of a sialic-acid-containing glycan treated by the previously described derivatization method specific to the linkage type of sialic acids can be determined by analyzing a sample which contains the sialic-acid-containing glycan, using an appropriate device, such as a matrix-assisted laser desorption/ionization (MALDI) time-of-flight mass spectrometer (TOFMS), and analyzing the thereby collected data by a computer. For example, Patent Literature 2 discloses a computer program for a glycan-structure analysis for determining the composition and structure of a glycan from data obtained for a glycan by mass spectrometry.

A sialic-acid-containing glycan treated by a derivatization method specific to the linkage type of sialic acids has a peak detected on a mass spectrum near a theoretical glycan mass-to-charge ratio calculated by increasing or decreasing the mass of the glycan by a different mass for each sialic-acid linkage type according to the kind of derivatization (this peak is hereinafter called the “ion peak”). For example, a derivatization method which can distinguish between α2,3 and α2,6 linkages increases the mass of the glycan by a different mass for each of the two linkage types. Accordingly, when N sialic acids are contained in one kind of glycan structure, a total of N+1 (=_{N+1}C_{N}) ion peaks having different mass-to-charge ratios may possibly be detected.

The computer program described in Patent Literature 2 determines the structure of a sialic-acid-containing glycan by determining the mass-to-charge ratios of the ion peaks in mass spectrum data obtained for a sample containing a sialic-acid-containing glycan treated with a derivatization method specific to the linkage type of sialic acids, and comparing those mass-to-charge ratios with a plurality of theoretical glycan mass-to-charge ratios previously set according to the derivatization method.

### CITATION LIST

### Patent Literature

Patent Literature 1: JP 2016-194500 A

Patent Literature 2: WO2020/079878 A

### Non Patent Literature

Non Patent Literature 1: N. de Haan, et. al., “Glycomics studies using sialic acid derivatization arid mass spectrometry”, Nature Reviews Chemistry, 2020, 4(5), 229-242

### SUMMARY OF INVENTION

### Technical Problem

In the of e ed computer program, a plurality of theoretical glycan mass-to-charge ratios are set on the assumption that all sialic acids of a predetermined linkage type in the sialic-acid-containing glycan will he derivatized, and those theoretical glycan mass-to-charge ratios are compared with the mass-to-charge ratios of the ion peaks in the mass spectrum data. However, depending on the conditions under which the sample is treated by the derivatization method specific to the linkage type of sialic acids, some of the sialic-acid-containing glycans contained in the sample may remain in their original form in which none of the sialic acids of the predetermined linkage type is derivatized, or in a form in which the sialic acids of the predetermined linkage type are not fully derivatized. Those sialic-acid-containing glycans will be present foreign substances in the sample. Foreign substances in a sample may possibly interfere with the analysis of glycan structures.

The present invention has been developed to solve the previously described problem. Its objective is to make it possible to recognize whether or not sialic-acid-containing glycans which have undergone a derivatization treatment specific to the linkage type of sialic acids have been derivatized as intended.

### Solution to Problem

A glycan mass spectrometry data analyzer according to the first aspect of the present invention developed for solving the previously described problem is a glycan mass spectrometry data analyzer configured to analyze mass spectrum data obtained by a mass spectrometric analysis of a sample containing a sialic-acid-linked glycan subjected to a derivatization treatment specific to a sialic-acid linkage type or a molecule modified with the sialic acid-linked glycan, the glycan mass spectrometry data analyzer including:
a glycan composition setting section configured to allow a setting of a glycan composition of an analysis target;
a glycan information acquisition section configured to acquire a plurality of kinds of glycans having a core structure formed by a plurality of monosaccharide residues of a plurality of kinds, and mass information corresponding to each of the plurality of kinds of glycans, based on the glycan composition set through the glycan composition setting section;
a mass-changing factor setting section configured to allow a setting of mass-changing factors causing a mass change of a glycan; and
a theoretical glycan mass calculation section configured to determine a theoretical glycan mass-to-charge ratio after the mass change of the glycan occurs, of each of the plurality of kinds of glycans acquired by the glycan information acquisition section, based on the mass-changing factors set through the mass-changing factor setting section,
where:
   the mass-changing factor setting section sets, as the mass-changing factors, an ion species or salt that is possibly formed on a carboxyl group of a sialic acid in a sialic-acid-linked glycan included in the analysis target, and a sialic-acid linkage type in which a mass change corresponding to the derivatization treatment possibly occurs; and
   the theoretical glycan mass calculation section includes a mass change calculation section configured to determine an amount of mass change estimated from the combination of the ion species or the salt and the sialic-acid linkage type.

The second aspect of the present invention developed for solving the previously described problem is a glycan-mass-spectrometry-data analyzing program fix analyzing mass spectrum data obtained by a mass spectrometric analysis of a sample containing a sialic-acid-linked glycan subjected to a derivatization treatment specific to a sialic-acid linkage type or a molecule modified with the sialic-acid-linked glycan, where the program is configured to make a computer function as:
a glycan composition setting section configured to allow a setting of a glycan composition of an analysis target;
a glycan information acquisition section configured to acquire a plurality of kinds of glycans having a core structure formed by a plurality of monosaccharide residues of a plurality of kinds, and mass information corresponding to each of the plurality of kinds of glycans, based on the glycan composition set through the glycan composition setting section
a mass-changing factor setting section configured to allow a setting of mass-changing factors causing a mass change of a glycan; and
a theoretical glycan mass calculation section configured to determine a theoretical glycan mass-to-charge ratio after the mass change of the glycan occurs, of each of the plurality of kinds of glycans acquired by the glycan information acquisition section, based on the mass-changing factors set through the mass-changing factor setting section,
where:
   the mass-changing factor setting section sets, as the mass-changing factors, an ion species or salt that is possibly formed on a carboxyl group of a sialic acid in a sialic-acid-linked glycan included in the analysis target, and a sialic-acid linkage type in which a mass change corresponding to the derivatization treatment possibly occurs; and
   the theoretical glycan mass calculation section includes a mass change calculation section configured to determine an amount of mass change estimated from the combination of the ion species or the salt and the sialic-acid linkage type.

### Advantageous Effects of Invention

In the case where a derivatization treatment specific to the linkage type of sialic acids has been performed on a glycan as an analysis target for the structural analysis of a sialic-acid-linked glycan, the present invention can provide users with information concerning, the mass-to-charge ratios of not only a plurality of glycans (glycan derivatives) resulting from the derivatization of the sialic acids of the original glycan and having different mass-to-charge ratios according to the difference in sialic-acid linkage type, but also foreign substances which are expected to be formed as a result of an incomplete or totally failed derivatization of the sialic acids. By comparing the information concerning a provided set of mass-to-charge ratios with mass spectrometry data (mass spectrum data), the user can determine whether or not the temperature, pH and other processing conditions for the derivatization treatment are appropriate.

### BRIEF DESCRIPTION OF DRAWINGS

FIG. 1 is a schematic block diagram of a glycan mass spectrometry data analyzing system as one embodiment of the present embodiment.

FIG. 2 is a flowchart of the procedure of an analytical processing in the glycan mass spectrometry data analyzing system according to the present embodiment.

FIG. 3 is an example of a tab which allows a user to set the kind of sialic-acid-linkage-specific derivatization in the glycan mass spectrometry data analyzing system according to the present embodiment.

FIG. 4 is an example of a tab which allows a user to set the glycan composition of an analysis target in the glycan mass spectrometry data analyzing system according to the present embodiment.

FIG. 5 is an example of a tab which allows a user to set ionization conditions in the glycan mass spectrometry data analyzing system according to the present embodiment.

FIG. 6 is an example of a tab which allows a user to set salt formation conditions in the glycan mass spectrometry data analyzing system according to the present embodiment.

FIG. 7 is an example of the mass-to-charge-ratio values of ion peaks specified as an analysis target.

FIG. 8 is an example of a mass spectrum obtained for a sample containing an analysis target.

### DESCRIPTION OF EMBODIMENTS

A glycan structure analyzing system as one embodiment of the present invention is hereinafter described with reference to the attached drawings.

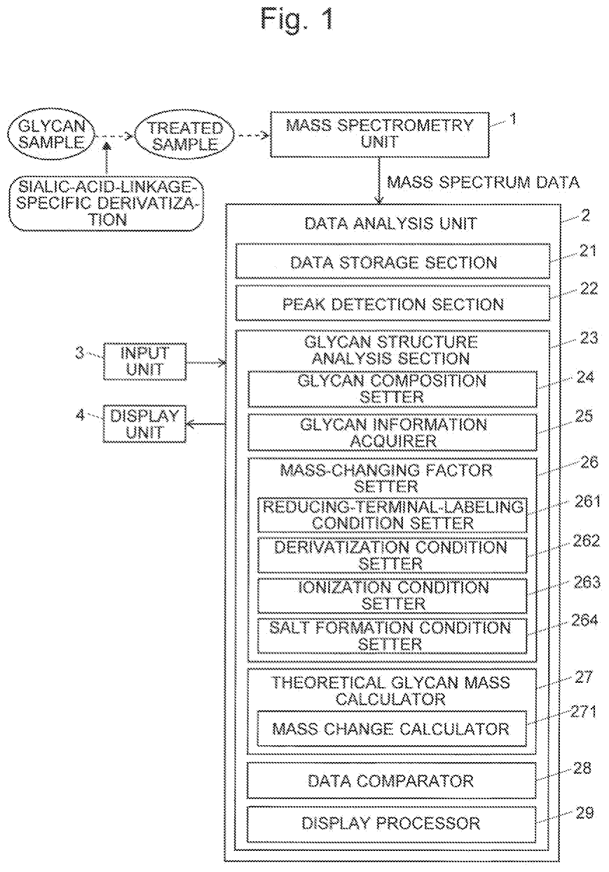

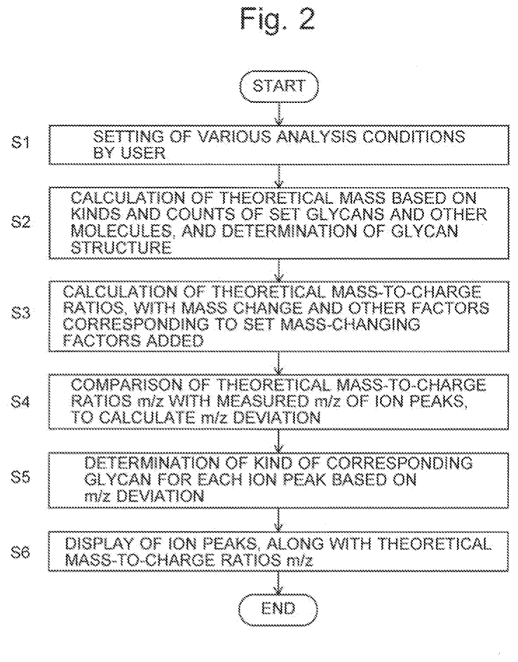

FIG. 1 is a schematic block diagram of the glycan structure analyzing system according to the present embodiment. As shown in FIG. 1, the present system includes a mass spectrometry unit 1 which performs a measurement on a sample, a data analysis unit 2 which performs an analytical processing, as well as an input unit 3 and a display unit 4 which serve as user interfaces. The data analysis unit 2 includes a data storage section 21, peak detection section 22 and glycan structure analysis section 23. The glycan structure analysis section 23 includes a glycan composition setter 24, glycan information acquirer 25, mass-changing factor setter 26, theoretical glycan mass calculator 27, data comparator 28 and display processor 29 as its functional blocks. The mass-changing factor setter 26 includes a reducing-terminal-labelling condition setter 261, derivatization condition setter 262, ionization condition setter 263 and salt formation condition seller 264 as its sub-functional blocks. The theoretical glycan mass calculator 27 includes a mass change calculator 271 as its sub-functional block.

The type of the mass spectrometry unit 1 is not specifically limited. In general, it should have a high level of mass accuracy and mass-resolving power. Therefore, for example, a time-of-flight mass spectrometer (TOFMS) or Fourier-transform ion cyclotron resonance mass spectrometer (FT-ICRMS) is useful. As for the ionization method in the mass spectrometer, the matrix-assisted laser desorption/ionization (MALDI) can be used. Other methods are also available, such as the electrospray ionization (ESI) or probe electrospray ionization (PESI), although the MALDI is preferable in that this method mainly generates singly-charged ions.

As will be described later, a normal mode of mass spectrometry which includes no dissociation of ions is often sufficient for the glycan structure analysis including the numbers and linkage types of the sialic acids modifying the glycan. However, for the structural analysis of a glycan linked to a peptide or similar molecule, it is common to perform an MSⁿ analysis with n being equal to or greater than two to acquire MSⁿ spectrum data. In that case, a mass spectrometer including an ion trap, collision cell or similar function for dissociating an ion by a collision induced dissociation (CID) or similar process is used as the mass spectrometry unit 1.

The data analysis unit 2 in the present system is actually a general-purpose personal computer or more sophisticated workstation, with the functions of the functional blocks shown in FIG. 1 realized by executing, on the computer, a dedicated data processing program installed on the same computer. This data processing program corresponds to the glycan structure analysis program according to the present invention. In that case, the input unit 3 includes a keyboard and pointing device (e.g., mouse) provided for the computer, while the display unit 4 includes the monitor provided for the same computer. The data processing program can be offered to users in the form of a non-transitory storage medium recording the program, such as a CD-ROM, DVD-ROM, memory card, or USB memory (dongle). It may also be offered to users in the form of data transferred through the Internet or similar communication networks.

When a glycan structure analysis is to be performed with the system according to the present embodiment, a sample containing a glycan (“glycan sample”) should be pretreated by a sialic-acid-linkage-specific derivatization, and the treated sample is subjected to mass spectrometry in the mass spectrometry unit 1 to acquire mass spectrum data covering a predetermined range of mass-to-charge ratios. As one example, it is hereinafter assumed that a sialic-acid-linkage-specific derivatization as described in Patent Literature 1 or Non Patent Literature 1 is performed in which the glycan sample is subjected to the reaction under the presence of a dehydration-condensation agent containing an amine and carbodiimide. In that case, if the sialic acid in the glycan is an α2,3-linked sialic acid. a lactone is formed as the derivative. If the sialic acid in the glycan is an α2,6-linked sialic acid, an amide is formed as the derivative. Although the glycans originally have the same composition, their derivatives differ from each other in mass. The mass spectrum data acquired in the mass spectrometry unit 1 for a sample which has undergone the previously described derivatization treatment is sent to the data analysis unit 2 and stored in the data storage section 21.

In the data analysis unit 2, the peak detection section 22 detects peaks in the collected mass spectrum data according to a predetermined algorithm, and acquires the mass-to-charge ratio and signal intensity of each peak to create a peak list. The created peak list is temporarily stored in the data storage section 21. This peak list will be the data to be analyzed by the glycan structure analysis section 23.

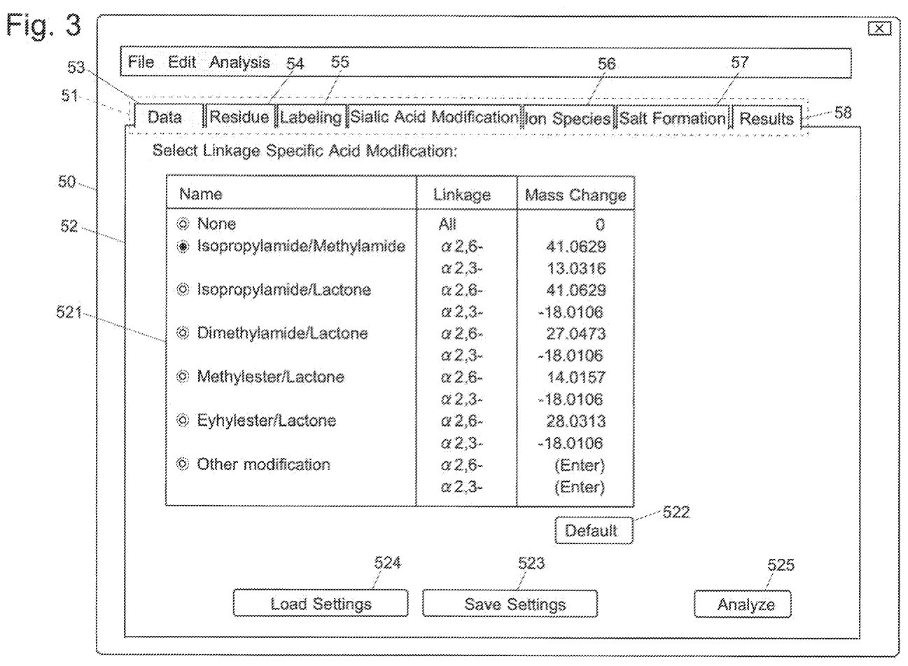

To carry out a glycan structure analysis, the user initially performs a predetermined operation from the input unit 3. In response to this operation, the glycan structure analysis section 23 displays a main window on the screen of the display unit 4. The main window has a plurality of switchable tabs. FIG. 3 shows the main window 50 in which one of the tabs, named the “Sialic Acid Modification” setting tab 52, is opened. Other than this “Sialic Acid Modification” setting tab 52, the window is provided with the “Data” setting tab 53, “Residue” setting tab 54, “Labelling” setting tab 55, “Ion Species” setting tab 56, “Salt Formation” setting tab 57, and result display tab 58 (“Results” tab in FIG. 3). The user can easily switch between these tabs by clicking a desired tab in a tab switching area 51.

Descriptions of those tabs are as follows:

The result display (“Results”) tab 58 is a tab on which the result of a glycan structure analysis will be displayed, while the other tabs allow a user to set analysis conditions before the execution of an analysis and issue a command to execute an analysis.

The “Data” setting tab 53 allows the user to select a peak list to he analyzed, and input numerical values, such as the allowable mass error of the measurement data. The inputted values determine the allowable error to be used when the mass-to-charge ratio of a measured ion peak is compared with theoretical glycan mass-to-charge ratios, as will be described later.

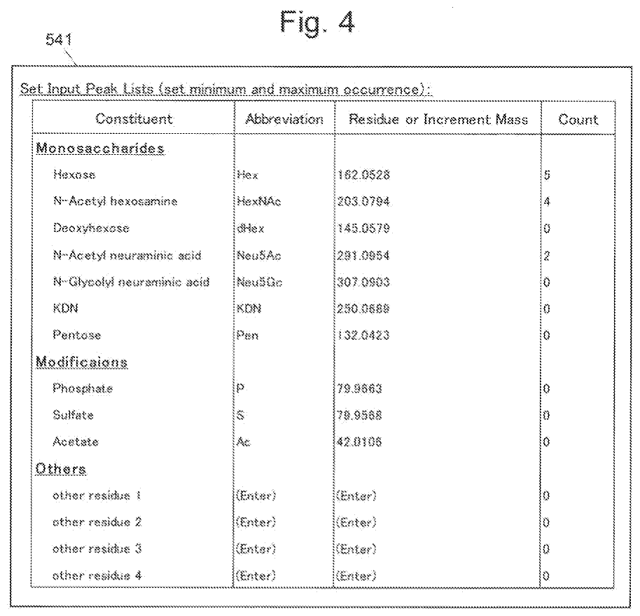

The “Residue” setting tab 54 allows the user to set a glycan composition which will be the analysis target. This tab is displayed by the glycan composition setter 24. FIG. 4 shows a setting target area 541 which is displayed in the main window 50 when the “Residue” setting tab 54 is opened. The setting target area 541 in the “Residue” setting tab 54 shows the names of sugars with their respective abbreviations and masses. In the present example, seven monosaccharides including sialic acids are shown: hexose, N-acetyl hexosamine, deoxyhexose. N-acetyl neuraminic acid, N-glycolyl neuraminic acid, KDN (2-keto-3-deoxy-D-glycero-D-galacto-nononic acid) and pentose. The same area 541 also shows the names of molecules which increases the mass of a glycan by modification (phosphate, sulfate and acetate) with their respective abbreviations and masses. Additionally, the area 541 allows for the setting of the count of each sugar or molecule. Setting a count value of zero means that the sugar or molecule is not included in the target glycan. Thus, the user can conveniently set a glycan composition by simply inputting the count of each kind of sugar or molecule included in the glycan as the analysis target.

If a sugar that is not displayed in the setting target area 541 is included in the glycan as the analysis target, the user should manually input the name, residue mass and count of that sugar. Thus, even when a rather uncommon sugar is included in the glycan, the user can easily set the glycan composition by simple operations.

The setting shown in FIG. 4 indicates that the glycan composition should include five hexoses, four N-acetyl hexosamines and two N-acetyl neuraminic acids.

The “Labelling” setting tab 55 allows the user to select the kind of modification by the labelling, a primary example of which is the fluorescent labelling. Though not shown, the “Labelling” setting; tab 55 includes a setting target area, in which, for example, 2-amino benzamide, pyridylamine or other labelling substances can be selected.

The “Sialic Acid Modification” setting tab 52 allows the user to select the kind of sialic-acid-linkage-specific derivatization. This tab is displayed by the derivatization condition setter 262. FIG. 3 shows the main window 50 in which the “Sialic Acid Modification” setting tab 52 is opened. The “Sialic Acid Modification” setting tab 52 has a setting target area 521 with radio buttons which allow the selection of one of the six options including the five preset kinds of sialic-acid-linkage-specific derivatizations and an undefined sialic-acid-linkage-specific derivatization (“Other modification” in FIG. 3). For each of the five preset sialic-acid-linkage-specific derivatizations, the amount of mass change (“Mass Change” in FIG. 3) based on a known change in chemical formula is defined for each of the two linkage types, i.e., α2,3 and α2,6 linkages. This allows the user to conveniently select preset values for derivatizations which are expected to be rather frequently used, such as the combination of the isopropyl amidation and methyl amidation published in the papers mentioned earlier. In the situation shown in FIG. 3, the combination of the isopropyl amidation and methyl amidation is selected as the sialic-acid-linkage-specific derivatization.

In the case of using an undefined derivatization reagent which is not included in the preset derivatizations, the user selects the “Other Modification” option and inputs a numerical value of the amount of mass change based on a change in chemical formula for each of the α2,3 and α2,6 linkages. This requires the user to perform only simple operations and proceed with the analysis even in the case of using a new or rather uncommon derivatization reagent which has not been assumed for use. It should be noted that clicking the “Default” button 522 on the “Sialic Acid Modification” setting tab 52 in FIG. 3 resets the information in the setting target area 521 to the default settings registered in a previously specified file.

When the “Save Settings” button 523 on the “Sialic Acid Modification” setting tab 52 is clicked by the user, the information which has been set in the setting target area 521 at that point in time is saved in a previously specified setting-information-saving file. On the other hand, When the “Load Settings” button 524 is clicked by the user, the information held in the setting-information-saving tile is automatically set in the setting target area 521. With this function, for example, the user only needs to perform a simple operation to conduct an analysis under the same condition as an analysis performed in the past.

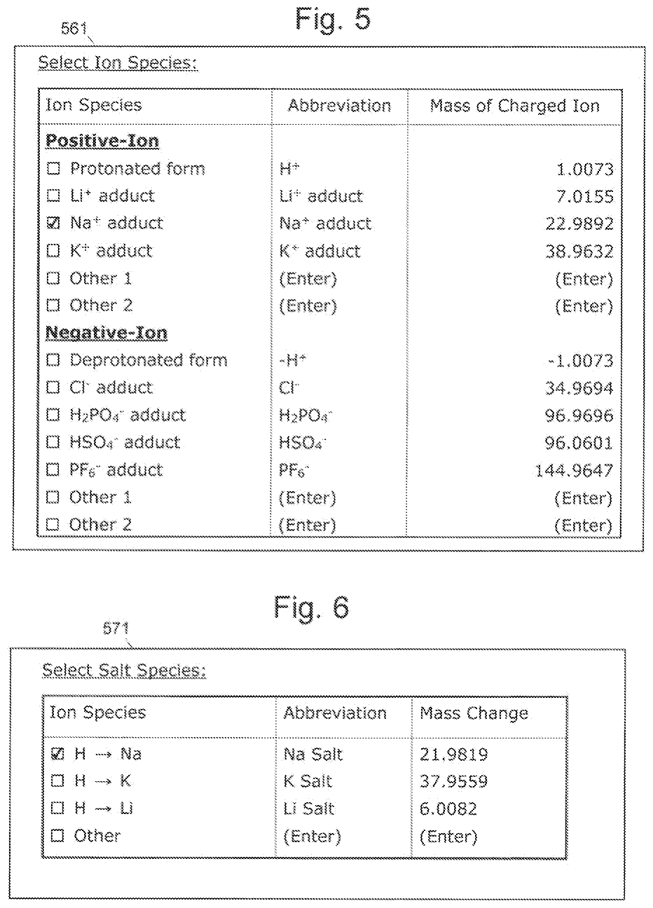

The “Ion Species” selling tab 56 allows the user to select the polarity, number of charges, and ion species of an ion produced by the ionization of the glycan. This tab is displayed by the ionization condition setter 263. FIG. 5 shows a setting target area 561 to be displayed in the main window 50 when the “Ion Species” setting tab 54 is opened. The setting target area 561 in the “Ion Species” setting tab 56 allows the selection of ion species from proton (H⁺) adduct, sodium ion (Na⁺) adduct or potassium ion (K⁺) adduct for positive ions, as well as proton (H⁻) elimination, chlorine ion (Cl⁻) adduct, phosphoric ion (H₂PO₄⁻) adduct, hydrogen sulfate ion (HSO₄⁻) adduct or hexafluorophosphate ion (PF₆⁻) adduct for negative ions. In the situation shown in FIG. 5. sodium ion (Na⁺) adduct is selected. The selling target area 561 also allows the user to additionally input the name and mass of an ion species so that the name of an ion species which is not included in the previously listed ion species can be selected.

The “Salt Formation” setting tab 57 allows the user to perform the setting of the substitution of a partial structure of the glycan that involves no change in the number of charges. This tab is displayed by the salt formation condition setter 264. FIG. 6 shows a setting target area 571 to be displayed in the main window 50 when the “Salt Formation” setting tab 57 is opened. The setting target area 571 allows the selection of the substitution of sodium, potassium or lithium for proton. In the situation shown in FIG. 6, the substation of sodium for proton is selected.

A procedure for analyzing glycan mass spectrometry data using the present system is hereinafter described.

A user appropriately sets analysis conditions in each tab of the main window 50 in the previously described manner (Step 1).

After the setting of the analysis conditions has been completed, the user clicks the “Analyze” button 525 on the “Sialic Acid Modification” setting tab 52 (or any other setting tab), The glycan structure analysis section 23 responds to this operation and actually initiates the analytical processing under the set analysis conditions.

That is to say, the glycan information acquirer 25 initially calculates the theoretical mass of the glycan based on the kinds and counts of the sugars and other related molecules expressing the glycan composition which has been set in the setting target area 541 of the “Residue” setting tab 54 (Step 2). Meanwhile, the glycan information acquirer 25 estimates the combination of the sialic-acid linkage types from the kinds and counts of the sugars and other related molecules expressing the glycan composition, and determines a plurality of glycan structures. For example, for a biantennary N-glycan which includes two sialic acids and all sialic-arid linkage types (this glycan is hereinafter called the “A2 glycan”), if a glycan consisting of five hexoses, four N-acetyl hexosamines and two N-acetyl neuraminic acids has been set as the glycan to be subjected to the sialic-acid-linkage-specific derivatization, the theoretical mass of the glycan is calculated by adding the theoretical trusses of hexose, N-acetyl hexosamine and N-acetyl neuraminic acid multiplied by their respective counts.

Subsequently, the theoretical glycan mass calculator 27 calculates a theoretical mass-to-charge ratio for each of the glycans having different structures on the assumption that the reducing-terminal labelling, derivatization, ionization and other conditions which have been set as the mass-changing factors have been performed (Step 3).

For example, consider the case where the labelling with 2-Aminobenzamide (AB-labelling, +120.0687 Da) has been selected as the condition of the labelling of the reducing-terminal of the glycan. Suppose also that the contents shown in the setting target areas in FIGS. 3, 5 and 6 have been set as the other mass-changing factors. That is to say, a derivatization reagent which induces methyl amidation of α2,3-linked sialic acids and isopropyl amidation of α2,3-linked sialic acids is selected as the sialic-acid-linkage-specific derivatization reagents; the ionization polarity is positive; and sodium ion (Na⁺) adduct is selected as the ion species. It should be noted that selecting the negative polarity as the polarity of the ionization will result in the generation of an anion, regardless of the selected ion species, due to the proton elimination which occurs on a carboxyl group that remains in a sialic acid due to an incomplete or totally failed derivatization.

When sodium salt has been selected as the salt formation condition, sonic of the carboxyl groups in the sialic acids which remain due to an incomplete or totally failed derivatization may possibly form sodium salts. For the present, it is assumed that the substitution of sodium for proton has been selected.

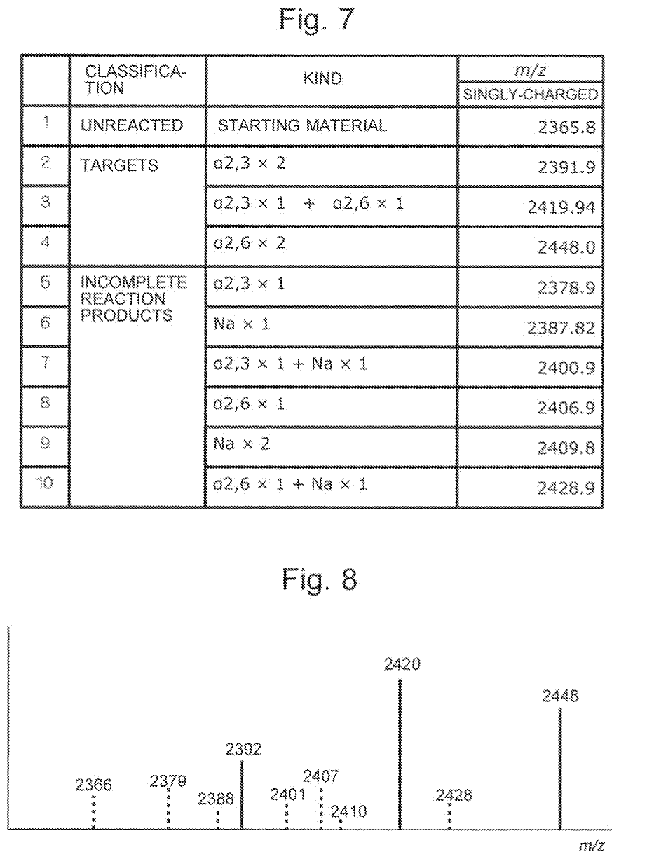

From the mass-changing factors thus set, the mass change calculator 271 calculates a mass-to-charge ratio for each of the four assumed situations in which the carboxyl group in each of the two sialic acids in the A2 glycan undergoes (1) no reaction, (2) substitution by sodium, (3) methyl amidation, provided that the sialic acid is an α2,3-linked sialic acid, or (4) isopropyl amidation, provided that the sialic acid is an α2,6-linked sialic acid. Specifically, a theoretical mass-to-charge ratio is calculated for each of a total of ten kinds of glycans (₂₊₄₋₁C₄₋₁), including one kind of unreacted glycan (i.e., the glycan as the starting material), three kinds of glycans as the intended products which have been fully derivatized, and six kinds of glycans which have resulted from incomplete derivatization (incomplete reaction products) FIG. 7 shows a list of the theoretical mass-to-charge ratios calculated in the case of a singly-charged ion.

Next, the data comparator 28 sequentially compares the mass-to-charge-ratio values (“measured values”) of the ion peaks in a given peak list with the theoretical mass-to-charge-ratio values (“theoretical values”) calculated in Step 3. In the present example, the deviation between the measured and theoretical values is computed for each ion peak (Step 4). If the deviation falls within a previously set permissible error, the ion peak is selected as the kind of glycan corresponding to the theoretical value (Step 5).

Subsequently, the display processor 29 creates a view which shows a mass spectrum in which the theoretical value of each kind of glycan determined as a result of the comparison by the data comparator 28 is added near the corresponding ion peak. When the result display tab 58 is opened by the user, the display processor 29 shows that view on the tab (Step 6).

FIG. 8 is a model diagram of a mass spectrum acquired with a matrix-assisted laser desorption/ionization mass spectrometer (MALDI-MS) for a sodium adduct ion of an AB-labeled A2 glycan which has undergone a sialic-acid-linkage-specific derivatization. As shown in FIG. 8, each ion peak has its theoretical m/z value displayed nearby. Among the displayed ion peaks, those located at m/z 2392. 2420 and 2448 correspond to the m/z values of the singly-charged ions of the intended products, while the ion peaks detected around the ion peaks of the intended products correspond to the m/z values of the singly-charged ions of the unreacted glycan and incomplete reaction products. It should be noted that, in FIG. 8, the ion peaks of the intended products are represented by solid lines, while those of the unreacted glycan and incomplete reaction products are represented by broken lines so as to help visually recognize the ion peaks of the unreacted glycan and incomplete reaction products occurred in addition to those of the intended products.

As described to this point, the glycan mass spectrometry data analyzing system according to the present embodiment allows the user to easily recognize that an unreacted glycan and/or incomplete reaction product has resulted from a derivatization treatment preformed for recognizing the linkage type of sialic acids. When, for example, the intensity of an ion peak corresponding to an unreacted glycan or incomplete reaction product is not negligible as compared to the intensity of the ion peak of an intended product, the user may consider reviewing the conditions of the derivatization treatment.

[Various Modes of Invention]

A person skilled in the art can understand that the previously described embodiment is a specific example of the following modes of the present invention.

(Clause 1) The first aspect of the present invention is a glycan mass spectrometry data analyzer configured to analyze mass spectrum data obtained by a mass spectrometric analysis of a sample containing a sialic-acid-linked glycan subjected to a derivatization treatment specific to a sialic-acid linkage type or a molecule modified with the sialic-acid-linked glycan, the glycan mass spectrometry data analyzer including:
a glycan composition setting section configured to allow a setting of a glycan composition of an analysis target;
a glycan information acquisition section configured to acquire a plurality of kinds of glycans having a core structure formed by a plurality of monosaccharide residues of a plurality of kinds, and mass information corresponding to each of the plurality of kinds of glycans. based on the glycan composition set through the glycan composition setting section;
a mass-changing factor setting section configured to allow a setting of mass-changing factors causing a mass change of a glycan; and
a theoretical glycan mass calculation section configured to determine a theoretical glycan mass-to-charge ratio after the mass change of the glycan occurs, of each of the plurality of kinds of glycans acquired by the glycan information acquisition section, based on the mass-changing factors set through the mass-changing factor setting section, where:
the mass-changing factor setting section sets, as the mass-changing factors, an ion species or salt that is possibly formed on a carboxyl group of a sialic acid in a sialic-acid-linked glycan included in the analysis target, and a sialic-acid linkage type in which a mass change corresponding to the derivatization treatment possibly occurs; and
the theoretical glycan mass calculation section includes a mass change calculation section configured to determine an amount of mass change estimated from the combination of the ion species or the salt and the sialic-acid linkage type.

(Clause 3) The second aspect of the present invention is a glycan-mass-spectrometry-data analyzing program for analyzing mass spectrum data obtained by a mass spectrometric analysis of a sample containing a sialic-acid-linked glycan subjected to a derivatization treatment specific to a sialic-acid linkage type or a molecule modified with the sialic-acid-linked glycan, where the program is configured to make a computer function as:
glycan composition setting section configured to allow a setting of a glycan composition of an analysis target;
a glycan information acquisition section configured to acquire a plurality of kinds of glycans having a core structure formed by a plurality of monosaccharide residues of a plurality of kinds, and mass information corresponding to each of the plurality of kinds of glycans, based on the glycan composition set through the glycan composition setting section;
a mass-changing factor setting section configured to allow a setting of mass-changing factors causing a mass change of a glycan; and
a theoretical glycan mass calculation section configured to determine a theoretical glycan mass-to-charge ratio after the mass change of the glycan occurs, of each of the plurality of kinds of glycans acquired by the glycan information acquisition section, based on the mass-changing factors set through the mass-changing factor setting section, where:
the mass-changing factor setting section sets, as the mass-changing factors, an ion species or salt that is possibly formed on a carboxyl group of a sialic acid in a sialic-acid-linked glycan included in the analysis target, and a sialic-acid linkage type in which a mass change corresponding to the derivatization treatment possibly occurs; and
the theoretical glycan mass calculation section includes a mass change calculation section configured to determine an amount of mass change estimated from the combination of the ion species or the salt and the sialic-acid linkage type.

In the case where a derivatization treatment specific to the linkage type of sialic acids has been performed on a glycan as an analysis target for the structural analysis of a sialic-acid-linked glycan, the present invention can provide users with information concerning the mass-to-charge ratios of not only a plurality of glycans (glycan derivatives) resulting from the derivatization of the sialic acids of the original glycan and having different mass-to-charge ratios according to the difference in sialic-acid linkage type, but also foreign substances which are expected to be formed as a result of an incomplete or totally failed derivatization of the sialic acids. By comparing the information concerning a provided set of mass-to-charge ratios with mass spectrometry data (mass spectrum data), the user can determine whether or not the temperature, pH and other conditions for the derivatization treatment are appropriate.

(Clause 2) The glycan mass spectrometry data analyzer described in Clause 1 may further include: a data-comparing section configured to determine whether or not an ion having a theoretical glycan mass-to-charge ratio calculated by the theoretical glycan mass calculation section is included in the mass spectrum data, by comparing the theoretical glycan mass-to-charge ratio with the mass spectrum data; and a comparison result display section configured to display a comparison result obtained by the data-comparing section.

(Clause 4) In the glycan-mass-spectrometry-data analyzing program described in Clause 3, the program may further make the computer function as: a data-comparing section configured to determine whether or not an ion having a theoretical glycan mass-to-charge ratio calculated by the theoretical glycan mass calculation section is included in the mass spectrum data, by comparing the theoretical glycan mass-to-charge ratio with the mass spectrum data; and a comparison result display section configured to display a comparison result obtained by the data-comparing section.

By using the glycan mass spectrometry data analyzer described in Clause 2 or the glycan-mass-spectrometry-data analyzing program described in Clause 4, the user can easily determine whether or not a glycan or molecule whose derivatization has been incomplete or totally failed is present among sialic-acid-linked glycans which have undergone a derivatization treatment specific to the linkage type of sialic acids or molecules modified by the glycan.

### REFERENCE SIGNS LIST

1 . . . Mass Spectrometry Unit

2 . . . Data Analysis Unit

21 . . . Data Storage Section

22 . . . Peak Detection Section

23 . . . Glycan Structure Analysis Section

24 . . . Glycan Composition Setter

25 . . . Glycan Information Acquirer

26 . . . Mass-Changing Factor Setter

261 . . . Reducing-Terminal-Labelling Condition Setter

262 . . . Derivatization Condition Setter

263 . . . Ionization Condition Setter

264 . . . Salt Formation Condition Setter

27 . . . Theoretical Glycan Mass Calculator

271 . . . Mass Change Calculator

28 . . . Data Comparator

29 . . . Display Processor

3 . . . Input Unit

4 . . . Display Unit

1. A glycan mass spectrometry data analyzer configured to analyze mass spectrum data obtained by a mass spectrometric analysis of a sample containing a sialic-acid-linked glycan subjected to a derivatization treatment specific to a sialic-acid linkage type or a molecule modified with the sialic-acid-linked glycan, the glycan mass spectrometry data analyzer comprising:
a glycan composition setting section configured to allow a setting of a glycan composition of an analysis target;
a glycan information acquisition section configured to acquire a plurality of kinds of glycans having a core structure formed by a plurality of monosaccharide residues of a plurality of kinds, and mass information corresponding to each of the plurality of kinds of glycans, based on the glycan composition set through the glycan composition setting section;
a mass-changing factor setting section configured to allow a setting of mass-changing factors causing a mass change of a glycan; and
a theoretical glycan mass calculation section configured to determine a theoretical glycan mass-to-charge ratio after the mass change of the glycan occurs, of each of the plurality of kinds of glycans acquired by the glycan information acquisition section, based on the mass-changing factors set through the mass-changing factor setting section,
where:
the mass-changing factor setting section sets, as the mass-changing factors, an ion species or salt that is possibly formed on a carboxyl group of a sialic acid in a sialic-acid-linked glycan included in the analysis target, and a sialic-acid linkage type in which a mass change corresponding to the derivatization treatment possibly occurs; and
the theoretical glycan mass calculation section includes a mass change calculation section configured to determine an amount of mass change estimated from a combination of the ion species or the salt and the sialic-acid linkage type.

2. The glycan mass spectrometry data analyzer according to claim 1, further comprising:
a data-comparing section configured to determine whether or not an ion having a theoretical glycan mass-to-charge ratio calculated by the theoretical glycan mass calculation section is included in the mass spectrum data, by comparing the theoretical glycan mass-to-charge ratio with the mass spectrum data; and
a comparison result display section configured to display a comparison result obtained by the data-comparing section.

3. A non-transitory computer readable medium recording a glycan-mass-spectrometry-data analyzing program for analyzing mass spectrum data obtained by a mass spectrometric analysis of a sample containing a sialic-acid-linked glycan subjected to a derivatization treatment specific to a sialic-acid linkage type or a molecule modified with the sialic-acid-linked glycan, wherein the program is configured to make a computer function as:
a glycan composition setting section configured to allow a setting of a glycan composition of an analysis target;
a glycan information acquisition section configured to acquire a plurality of kinds of glycans having a core structure formed by a plurality of monosaccharide residues of a plurality of kinds, and mass information corresponding to each of the plurality of kinds of glycans, based on the glycan composition set through the glycan composition setting section;
a mass-changing factor setting section configured to allow a setting of mass-changing factors causing a mass change of a glycan; and
a theoretical glycan mass calculation section configured to determine a theoretical glycan mass-to-charge ratio after the mass change of the glycan occurs, of each of the plurality of kinds of glycans acquired by the glycan information acquisition section, based on the mass-changing factors set through the mass-changing factor setting section,
where:
the mass-changing factor setting section sets, as the mass-changing factors, an ion species or salt that is possibly formed on a carboxyl group of a sialic acid in a sialic-acid-linked glycan included in the analysis target, and a sialic-acid linkage type in which a mass change corresponding to the derivatization treatment possibly occurs; and
the theoretical glycan mass calculation section includes a mass change calculation section configured to determine an amount of mass change estimated from a combination of the ion species or the salt and the sialic-acid linkage type.

4. The medium recording a glycan-mass-spectrometry-data analyzing program according to claim 3, wherein the program is further configured to make the computer function as:
a data-comparing section configured to determine whether or not an ion having a theoretical glycan mass-to-charge ratio calculated by the theoretical glycan mass calculation section is included in the mass spectrum data, by comparing the theoretical glycan mass-to-charge ratio with the mass spectrum data; and
a comparison result display section configured to display a comparison result obtained by the data-comparing section.
